(19) United States
(12) Patent Application Publication
(10) Pub. No.: US 2017/0176459 A1
(43) Pub. Date: Jun. 22, 2017

(54) NEUROTOXICITY BIOMARKERS FOR DIAGNOSIS AND TREATMENT OF ACUTE TRAUMATIC BRAIN AND SPINAL CORD INJURY

(71) Applicant: GRACE LABORATORIES, LLC, Atlanta, GA (US)

(72) Inventors: Svetlana Dambinova, Atlanta, GA (US); Alexander Skoromets, Atlanta, GA (US)

(73) Assignee: GRACE LABORATORIES, LLC, Atlanta, GA (US)

(21) Appl. No.: 15/388,209

(22) Filed: Dec. 22, 2016

Related U.S. Application Data
(60) Provisional application No. 62/270,696, filed on Dec. 22, 2015.

(51) Int. Cl. G01N 33/68; A61K 38/06

(52) U.S. Cl. CPC G01N 33/6896; A61K 38/06; G01N 33/6875; G01N 2333/705; G01N 2800/28

(57) ABSTRACT

Provided herein are compositions, methods and kits for determining the level of an AMPAR/KAR peptides in a subject. The methods include obtaining a biological sample from the subject and determining a level of one or more AMPAR/KAR peptides selected from the group consisting of GluR1/GluR6_7, GluR2/GluR6_7, GluR3/GluR6_7, GluR1/GluR6, GluR2/GluR7, and any combination thereof in the biological sample.

### CROSS-REFERENCES TO RELATED APPLICATION

This application claims priority to U.S. Provisional Application No. 62/270,696, filed Dec. 22, 2015, which is incorporated by reference herein in its entirety.

### BACKGROUND

Each year more than 3 million people sustain traumatic brain injury (TBI). McAllister, in Traumatic Brain Injury (2 ed.), ed. J. M. Silver, T. W. McAllister and S. C. Yudofsky, American Psychiatric Publishing, Inc., Arlington, Va., 2011, p. 239. In this population acute mild TBI is a major health problem, especially among individuals of ages 15 to 24 years. Sports and recreational activities contribute to approximately 21% of all cases of TBI. Recent epidemiologic data suggest more than 62,000 cases per year occur in high school contact sports. See Powell, Barber-Foss, JAMA, 1999, 282, 958. Thirty-four percent of U.S. college football players have experienced at least one concussion and 20% have experienced multiple concussions. Luckstead, AAP Grand Rounds, 2004, 11, 16. Sports-related concussions offer an important research opportunity to investigate the utility of early biomarkers for mild TBI because the time of head injury is known and blood samples can be drawn within 24 hours.

Immediate, secondary and cumulative consequences follow mild TBI. Cerebral edema and associated increased intracranial pressure are the major immediate consequences of TBI. Traumatized brains also have increased sensitivity to secondary ischemic insult, which is triggered by a neurotransmitter storm evoked by the impact. Jain, Drug Discovery Today, 2008, 13, 1082. The cumulative incidence of mild TBI may result in post-traumatic epilepsy (PTE), which occurs in 4.4 per 100 persons with mild TBI in the first 3 years after hospital discharge. Ferguson et al, Epilepsia, 2010, 51, 891.

Acute subclinical concussions may be associated with microscopic widespread neurotoxicity and loss of neuronal connectivity (reversible changes), that may follow by minor ischemic or hemorrhagic complications due to compression or decompression of microvessels in nervous tissue. McAllister, in Traumatic Brain Injury (2 ed.), ed. J. M. Silver, T. W. McAllister and S. C. Yudofsky, American Psychiatric Publishing, Inc., Arlington, Va., 2011, p. 239. Normally, this subtle cell impairment is reversible and not visible on conventional CT and Mill images. Detection of impairment requires advanced radiological methods to recognize such abnormalities in grey or white matter. To date only the 9.4 Tesla Mill is able to observe metabolic processes in a single neuron. Atkinson, Lu, Thulborn, Magn. Reson. Med., 2011, 66, 1089. However, the 9.4 Tesla is currently only used in research mode and is not yet available for clinical assessments.

In the past five years, several published reports have found that diffusion tensor imaging (DTI) is sensitive to alterations of axonal shear and stretch in white matter caused by impact, that has been shown to correlate with severity of mild TBI and neuropsychological deficits. Due to sensitivity to iron and deoxygenated hemoglobin, Susceptibility Weighted Imaging (SWI) has demonstrated detection of hemorrhages, including micro-hemorrhages frequently seen in mild TBI. Used together, these advanced imaging techniques have the potential to serve as a set of surrogate biomarkers that can be used in diagnosis of mild TBI to triage impacted subjects for emergent treatment. To date, however, such imaging techniques are unavailable on regular basis or unreliable in some points for diagnosing the occurrence or severity of mild TBI.

### BRIEF SUMMARY

The present application relates to assessment of acute brain damage after an impact performed in biological samples drawn from subjects using rapid assays that detect neurotoxicity biomarkers, namely the family of non-NMDAR glutamate receptors. These receptors have an important diagnostic and prognostic capabilities to triage persons with mild TBI for emergent treatment navigation. Provided herein are compositions, methods and kits for determining the level of AMPAR/KAR peptides in a subject. The methods include obtaining a biological sample from the subject and determining a level in the biological sample of one or more AMPAR/KAR peptides selected from the group consisting of GluR1/GluR6_7, GluR2/GluR6_7, GluR3/GluR6_7, GluR1/GluR6, GluR2/GluR7, and any combination thereof. The kits include binding agents capable of binding to a substance within a biological sample, wherein said substance is an AMPAR/KAR peptide selected from the group consisting of SEQ ID NO:6, SEQ ID NO:7, SEQ ID NO:8, SEQ ID NO:9, SEQ ID NO:10, any combination thereof and a detecting reagent or a detecting apparatus capable of detecting binding of said binding agent to said substance. Optionally, the binding agents are antibodies against these peptides. Optionally, the kits include AMPAR/KAR peptides selected from the group consisting of SEQ ID NO:6, SEQ ID NO:7, SEQ ID NO:8, SEQ ID NO:9, SEQ ID NO:10, and any combination and/or antibodies against said peptides thereof.

The details of one or more embodiments are set forth in the accompanying drawings and the description below. Other features, objects, and advantages will be apparent from the description and drawings, and from the claims.

### BRIEF DESCRIPTION OF THE DRAWINGS

FIGS. 1A, 1B, and 1C are T2-weighted magnetic resonance imaging (MM) images of spinal cords. FIG. 1A shows the image of a patient (male, 47 y.o.) with anterior spinal artery syndrome based on hypercoagulation (SERPINE1 gene mutation), 40 days after onset, lower paraplegia, sensory loss, sphincter function loss. AMPAR/KAR peptide concentrations of 3.54 ng/ml were detected. FIG. 1B is an image of a patient (female, 52 y.o.) with spinal cord injury (SCI) after episode of hypotension, 2 years after the first onset, lower mild paraparesis. AMPAR/KAR peptide concentrations of 2.41 ng/ml were detected. FIG. 1C is an image of a patient (male, 53 y.o.) suffering from disc herniation, 9 months after onset, mild pain and vibration sensory loss. AMPAR/KAR peptide concentrations of 1.77 ng/ml were detected.

FIGS. 2A, 2B, 2C and 2D are diffusion tensor imaging (DTI) scans of an athlete one (1) year after last concussion. Three-dimensional DTI images with Fractional Anisotropy (FA) map generated with syngo DTI Tractography for a healthy person are shown in FIG. 2A and for a subject with concussion are shown in FIG. 2B. White matter fiber tracts seen with syngo DTI Tractography (256 diffusion directions) in a healthy person are shown in FIG. 2C and in a subject with concussion are shown in FIG. 2D. Arrows pointed to areas of injury.

FIGS. 3A, 3B and 3C are images of immunohistochemical staining by use of specific antibodies against AMPAR (green) and KAR (blue) peptides performed in rat spinal cord injury. Vehicle treatment is shown in FIG. 3A. The 3rd day after pretreatment with 30 μL Gafargin (the commercial name for the composition comprising the peptide Gly-Arg-Glu plus the chealating agent iron (Fe) (Grace Laboratories, LLC, Atlanta, Ga.)) demonstrated reduced axonal dieback. Gafargin showed neurotrophic effect in long-term live rat hippocampal slices (FIG. 3C).

FIG. 4 are graphs showing high performance liquid chromatography (HPLC) of cerebrospinal fluid (CSF) drawn from cistern magna of patients with mild TBI (n=33) before and after intranasal administrations of Gafargin (the commercial name for the composition comprising the peptide Gly-Arg-Glu plus the chealating agent iron (Fe) (Grace Laboratories, LLC, Atlanta, Ga.)), given at 0.1 mg daily for 1 week, 10 drops per day, 5 drops in each nostril). 1—before treatment; 2—24 hours after treatment; 3—after 3 days of treatment.

### DETAILED DESCRIPTION

Reliable and cost-effective biomarkers are useful to establish a diagnosis of TBI, especially in those with mild cases, where the impact often goes undetected. In patients who have no immediate symptoms or have contraindications related to brain MM, biomarkers can improve diagnostic certainty, allowing timely identification and triaging for personalized treatment. Furthermore, in the acute setting and during early clinical follow-up, biomarkers could be useful to assess the impact of or determine potential complications, such as the development of cerebral edema or ischemia that can follow mild TBI. Biomarkers could also serve useful in identifying individuals who are at risk for second impact syndrome, guiding the decision of when someone who has suffered a mild TBI can safely assume his/her activity.

Diagnosis of concussion is complicated because many primary concussions go unrecognized or they are not reported. This situation is especially true when a concussion is related to a sport injury, where loss of consciousness is rare. Additionally, competitive athletes are often subjected to recurrent concussions. If left unrecognized, such concussions may lead to potentially more debilitating delayed second-impact injuries.

Currently available blood-borne biomarkers that indicate brain injury, e.g., S-100 (Bazarian et al, Brain Inj., 2006, 20, 759), NSE (Geyer et al, J. Neurosurg. Pediatr., 2009, 4, 339), and cleaved Tau protein (Bazarian et al, Brain Inj., 2006, 20, 759), are still in the research phase and have not yet demonstrated satisfactory sensitivity or specificity in recognizing asymptomatic cases of concussions (Davis et al, Br. J. Sports Med., 2009 43 (Suppl I), i36). The excitatory neurotransmitter receptors as biomarkers of neurotoxicity for acute and chronic conditions of brain injury represent a promising avenue for selection of preventive treatment and follow up. Blaylock, Maroon, Surg. Neurol. Inter., 2011, 2, 107. The latter raises three significant questions: (i) for injured athletes, is prolonged resting after a concussion adequate to overcome consequences of multiple impacts?; (ii) should neuroprotective treatment be administered to “perfectly” healthy young athletes?; and (iii) if so, how do you determine the necessity of such treatment?

Optionally, as to existence of a concussion, additional investigations combining results from advanced Mill, neurotoxicity biomarker assays, and neurocognitive testing may be further utilized to improve diagnostic certainty of acute and chronic concussions.

Concussion or minor impact head injury is a brief episode of focal and/or diffuse neurological dysfunction that involves postconcussive symptoms and altered mental status. Davis et al, Br. J. Sports Med., 2009 43 (Suppl I), i36. Confusion, posttraumatic amnesia, visual problems, ringing in the ears, and headache are common signs after acute concussions. However, some cases, especially primary ones, may not have any clinical signs or symptoms for several days or weeks. Davis et al, Br. J. Sports Med., 2009 43 (Suppl I), i36.

The immediate damage due to severe or moderate concussions is recognizable and can be confirmed by brain imaging, namely, conventional computerized tomography (CT) scan. However, when diagnosing concussions or mild traumatic brain injury (TBI), CT and Mill scans are often negative. There is no objective evidence that mild impact leads to structural damage that can be viewed with such imaging techniques; however, it can trigger metabolic and functional disturbances, causing development of long-term secondary impairment in nervous tissue. There is a critical need to develop a method for early recognition of the brain metabolic and functional disturbances and to predict/prevent worsening of these abnormalities. The goal of such a test would be to aid in selection and prompt administration of neuroprotective therapy that will delay or reverse secondary damage after the neurotrauma.

Approximately one third of the brain is devoted to the mechanics of vision and visual processing. Kandel et al, in: Principles of Neural Science (4 ed.), ed. E. R. Kandel, J. H. Schwartz and T. M. Jessell, McGraw-Hill, New York, 2000, p. 533. Many concussions, especially coup-conter-coup injuries, can result in focal or diffuse axonal injuries that affect temporary memory loss, migraine, and abnormal spiking activity on EEG.

Minor impact stimulates a molecular chain reaction of neurotoxicity in which long-term depolarization and dysfunction in signal transduction evokes brief neuronal firing leading to risk of posttraumatic headache or abnormal spiking activity (Yasseen, Colantonio, Ratcliff, Brain Inj. 2008, 22, 752; Seifert, Evans, Curr. Pain Headache Rep., 2010, 14, 292). Kandel et al, p. 533. Ionotropic receptors (AMPA, kainite) cause an opening of channels, allowing potassium to flow out and calcium and sodium to enter (Betzen et al, Free Radical Biol. Med., 2009, 47, 1212). The AMPA receptor subunits are located exclusively in synaptic terminals and could indicate diffuse dendrite-axonal injury. AMPAR is primarily distributed in the forebrain and subcortical pathways, including the hippocampus, amygdala, thalamus, hypothalamus, and brain stem. Dambinova et al. Frontiers in Neurology, 2016, https://doi.org/10.3389/fneur.2016.00172.

Kainate receptor families are located mostly in the hippocampus, subcortical areas, spinal cord tract, and brainstem, might potentially affect cerebral venous circulation. Glutamate serves as a neuromediator for the medulla involved in regulation of involuntary life sustaining functions, such as breathing, swallowing, heart rate, and consciousness, primarily through AMPAR/KAR. In patients with mild TBI, the decrease of venous function due to a rise in venous oxygenation in the affected thalamostriate and right basal areas might involve KAR.

Mild TBI is the most prevalent form of head injury in civilian and military settings. Diagnosis of mild TBI poses some difficulties, particularly challenges in determining consequences after the injury. As a multi-factorial condition, TBI is manifested throughout the continuum of care, causing risk of development in future posttraumatic epilepsy (PTE), stroke, and other neurological conditions. Ferguson et al, Epilepsia, 2010, 51(5), 891; Chen, Kang, Lin, Stroke, 2011, 42(10), 2733.

Accurate and early identification of concussions and mild TBI using brain biomarker assays, optionally in conjunction with advanced neuroimaging, has the potential to reduce the number of undiagnosed cases, which, if left untreated, can lead to more debilitating and even fatal second-impact injuries. Cernak, Noble-Nacusslein, J. Cereb. Blood Flow and Metab., 2010, 30(2), 255. Such confirmation may also assist physicians in determining readiness of a person to return to work, duty, and play after a concussion. This approach might help triage persons seeking immediate neuroprotective treatment to prevent secondary injuries. Jain, Drug Discov. Today, 2008, 13(23-24), 1082.

Neurotoxicity biomarker blood tests may also have the potential to stratify risk of possible consequences after concussions and mild TBI. These assays serve as prognostic tools to assess the degree of brain injury causing epileptiform activity or/and cerebrovascular accident, or stroke. Thus, the assays have a significant impact on patient care by assisting in diagnosis and management of patients with brain injuries.

Mild TBI is the net effect of chronic encephalopathy that may be temporary (primary) concussions, long lasting (secondary), or even result in a permanent disruption of neuronal connectivity in one or more regions of the brain. Dambinova, in: Therapeutic Electrical Stimulation of Human Brain & Nerves, ed. N. P. Bechtereva, Soya, St. Petersburg, 2008, p. 346. In addition to impaired oxidative metabolism (Zazulia, Videen, Powers, Stroke, 2009, 40, 1638), the posttraumatic neurometabolic cascade may include hyper glycolysis, accumulation of lactate, enzyme-activated apoptosis, disrupted cytoskeletal architecture, axon swelling and secondary axotomy, free radical production and inflammation, impaired connectivity and altered neurotransmission, and altered cerebral blood flow (Barkhoudarian, Hovda, Giza, Clin. Sports Med., 2011, 30, 33). These structural and metabolic abnormalities may be detected by advanced radiological methods (e.g., diffusion tensor imaging, functional Mill, and DWI), Davis et al, Br. J. Sports Med., 2009 43 (Suppl I), i36).

Secondary pathological events underlie multiple mild TBI initiated by energy failure and protein synthesis/degradation processes. Following injury, blood flow is altered (Vespa, Stroke, 2009, 40, 1547; Qureshi et al, Crit. Care Med., 2003, 31, 1482), resulting in neurotoxicity (excitotoxicity) that leads to cell death through apoptosis or necrosis. It is known that AMPA receptors are mostly present in axonal and dendritic processes of the frontal lobe, hippocampus, amygdala, and cerebellum. In cerebellar Purkinje cells, which normally lack the NMDA receptor (Renzi et al, J. Physiol., 2007, 585, 91), the majority of synaptic contacts contain AMPA/kainite receptors. NMDA receptor density has been found presumably on microvessel surfaces (e.g., NR2 receptors)(Sharp et al, BMC Neurosci., 2003, 4, 28) and in gray matter (NR1 receptors, Nagy et al, Eur. J. Neurosci., 2004, 20, 3301) and is engaged in microvessel function and synaptic signal transduction (NR1/NR2 receptors) in the retina (Connaughton, in: Glutamate Receptors in Peripheral Tissue: Excitatory Transmission Outside the CNS, ed. G. Santokh and O. Pulido, Kluwer Academic/Plenum Publishers, New York, 2010, p. 99). Kainate receptors are present in brain stem and spinal cord axons and interact with specific mitochondrial metabolites. Rozas, in Kainate Receptors: Novel Signaling Insights (Advances in Experimental Medicine and Biology), ed. A. Rodriguez-Moreno and T. S. Sihra, Springer, New York, 2011, p. 72. Therefore, ionotropic glutamate receptors may be associated with certain locations of the injury and related to specific subcortical/axonal symptom presentation. Zazulia, Videen, Powers, Stroke, 2009, 40, 1638.

Chronic encephalopathy, as a secondary long-term consequence of mild TBI, confers difficulties in concentration during daily activity, sleep problems, and headaches. Increased likelihood of stroke, epilepsy, and brain tumors caused by neuronal impairment can be correlated with the area of brain structural damage and may lead to permanent disabilities with enduring neurotoxic damage. Chen, Kang, Lin, Stroke, 2011, 42, 2733; Zazulia, Videen, Powers, Stroke, 2009, 40, 1638.

Early diagnosis of mild TBI is critical to prevent secondary irreversible events occurring in the brain. A diagnosis of subtle damages will allow tailoring of timely, appropriate treatment to prevent neurotoxic damages, which can lead to a number of neurological conditions. Chen, Kang, Lin, Stroke, 2011, 42, 2733; Dambinova, in: Therapeutic Electrical Stimulation of Human Brain & Nerves, ed. N. P. Bechtereva, Soya, St. Petersburg, 2008, p. 346.

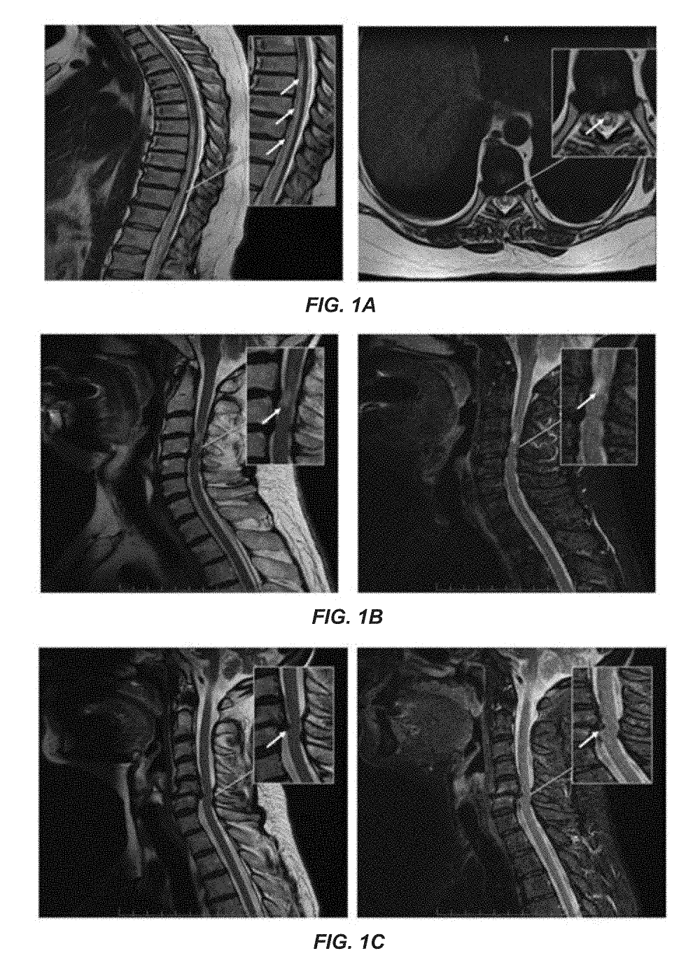

Provided herein are compositions, methods and kits to identify the anatomical location of AMPAR and kainite receptor over-expression in a human brain and spinal cord, based on the receptors subunit or isoform that is detected after the injury. Also provided are markers for determining neurotoxicity due to trauma to CNS such as concussions, mild traumatic brain or incomplete spinal cord injury. The provided compositions, methods and kits can distinguish mild TBI/incomplete SCI based on over-expression of the AMPA and kainate receptor, and the receptors subunit/isoform detected in bodily fluids (See, e.g., the examples and FIGS. 1A, 1B, and 1C). The provided compositions, methods and kits allow for assessment of CNS injury using panel of AMPAR/KAR biomarkers in conjunction with neurological/neurosurgical observations and advanced neuroimaging. Also provided are therapies that are useful for treating and preventing secondary injury in CNS after mild TBI or incomplete SCI based on the results of AMPA/kainite receptor fragments detection and quantification in bodily fluids.

Provided herein are strategies for diagnosing and for providing early treatment of neurotoxic events in the central nervous system that are caused by mild trauma to the brain or spinal cord. It has been discovered that AMPA/kainite receptors play a role in mild traumatic brain and incomplete spinal cord injuries, and it has been further discovered that these injuries give rise to detectable levels of particular AMPA/kainite receptor markers in the bloodstream. In particular, mild TBI and incomplete spinal cord injuries often lead to secondary ischemic events and abnormal brain spiking activity, if not treated properly. The markers provided herein are probative of these secondary and downstream events. In essence, particular recombinant and/or mutant AMPAR and GluR6/7 peptides are over-expressed in different anatomical regions of the CNS, and, when these subunits are over-expressed, they are proteolysed into peptide fragments that enter the bloodstream and are recognized as foreign antigens by the immune system, which responds by generating detectable amounts of antibodies. The existence and location of the above-mentioned subunit over-expressions in the CNS can thus be ascertained by detecting AMPAR and GluR6/7 isoform fragments or antibodies in bodily fluids such as the bloodstream, and by determining the particular subunit or isoform of AMPAR and GluR6/7 that is present. Still other embodiments pertain to an inhibitor that selectively prevents neurotoxicity and/or promotes neuronal survival, defined by chemical structure (I): Gly-X-Glu, wherein (a) Gly is the residue of glycine and Glu is the residue of glutamic acid; and (b) X is a chain comprising from two to four amino acid residues. The inhibitor may, optionally, include a chelating agent selected from magnesium, iron and zinc. Thus, provided are methods for inhibiting neurotoxicity by administering a compound defined by chemical structure (I) to a subject. Also provided are pharmaceutical compositions that contain the compound defined by chemical structure (I). Also provided are methods for assessment of subtle CNS injury (asymptomatic) using panel of AMPAR/KAR biomarkers in conjunction with neurological/neurosurgical observations and advanced neuroimaging.

As used herein, the singular forms a, an and the include plural referents unless the context clearly dictates otherwise. Thus, for example, reference to “a fragment” includes mixtures of fragments, reference to “an cDNA oligonucleotide” includes more than one oligonucleotide, and the like.

Protein and peptide are used interchangeably herein and include a molecular chain of amino acids linked through peptide bonds. The terms do not refer to a specific length of the product. Thus, peptides, oligopeptides, and proteins are included within the definition of polypeptide. The terms include post-translational modifications (isoforms) of the polypeptide, for example, glycosylations, acetylations, phosphorylations, chelates, and the like. In addition, protein fragments, analogs, mutated or variant proteins, chimeric peptides and the like are included within the meaning of polypeptide. The polypeptide, protein and peptides may be in cyclic form or they may be in linear form. In one particular embodiment, the peptides of the current invention are deglycosylated, or dephosphorylated from their natural state, or are prepared synthetically without naturally occurring glycosylation or phosphorylation.

Nucleic acid, as used herein, refers to deoxyribonucleotides or ribonucleotides and polymers and complements thereof. The term includes deoxyribonucleotides or ribonucleotides in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, conservatively modified variants of nucleic acid sequences (e.g., degenerate codon substitutions) and complementary sequences can be used in place of a particular nucleic acid sequence recited herein. Specifically, degenerate codon substitutions may be achieved by generating sequences in which the third position of one or more selected (or all) codons is substituted with mixed-base and/or deoxyinosine residues. Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

A nucleic acid is operably linked when it is placed into a functional relationship with another nucleic acid sequence. For example, DNA that encodes a presequence or secretory leader is operably linked to DNA that encodes a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, operably linked means that the DNA sequences being linked are near each other, and, in the case of a secretory leader, contiguous and in reading phase. However, enhancers do not have to be contiguous. For example, a nucleic acid sequence that is operably linked to a second nucleic acid sequence is covalently linked, either directly or indirectly, to such second sequence, although any effective three-dimensional association is acceptable. A single nucleic acid sequence can be operably linked to multiple other sequences. For example, a single promoter can direct transcription of multiple RNA species. Linking can be accomplished by ligation at convenient restriction sites. If such sites do not exist, the synthetic oligonucleotide adaptors or linkers are used in accordance with conventional practice.

The terms identical or percent identity, in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be substantially identical. This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A comparison window, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981); by the homology alignment algorithm of Needleman & Wunsch, J. Mol. Biol. 48:443 (1970); by the search for similarity method of Pearson & Lipman, Proc. Nat'l. Acad. Sci. USA 85:2444 (1988); by computerized implementations of these algorithms (GAP, BESTFIT, FASTA, and TFASTA in the Wisconsin Genetics Software Package, Genetics Computer Group, 575 Science Dr., Madison, Wis.); or by manual alignment and visual inspection (see, e.g., Current Protocols in Molecular Biology (Ausubel et al., eds. 1995 supplement)).

A preferred example of an algorithm that is suitable for determining percent sequence identity and sequence similarity are the BLAST and BLAST 2.0 algorithms, which are described in Altschul et al., Nuc. Acids Res. 25:3389-3402 (1977), and Altschul et al., J. Mol. Biol. 215:403-410 (1990), respectively. BLAST and BLAST 2.0 are used, with the parameters described herein, to determine percent sequence identity for nucleic acids or proteins. Software for performing BLAST analyses is publicly available through the National Center for Biotechnology Information, as known in the art. This algorithm involves first identifying high scoring sequence pairs (HSPs) by identifying short words of a selected length (W) in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The Expectation value (E) represents the number of different alignments with scores equivalent to or better than what is expected to occur in a database search by chance. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, expectation (E) of 10, and the BLOSUM62 scoring matrix (see Henikoff & Henikoff, Proc. Natl. Acad. Sci. USA 89:10915 (1989)), alignments (B) of 50, expectation (E) of 10, M=5, N=−4, and a comparison of both strands.

The term polypeptide, as used herein, generally has its art-recognized meaning of a polymer of at least three amino acids and is intended to include peptides and proteins. However, the term is also used to refer to specific functional classes of polypeptides, such as, for example, desaturases, elongases, etc. For each such class, the present disclosure provides several examples of known sequences of such polypeptides. Those of ordinary skill in the art will appreciate, however, that the term polypeptide is intended to be sufficiently general as to encompass not only polypeptides having the complete sequence recited herein (or in a reference or database specifically mentioned herein), but also to encompass polypeptides that represent functional fragments (i.e., fragments retaining at least one activity) of such complete polypeptides. Moreover, those in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term polypeptide as used herein. Those in the art can determine other regions of similarity and/or identity by analysis of the sequences of various polypeptides described herein. As is known by those in the art, a variety of strategies are known, and tools are available, for performing comparisons of amino acid or nucleotide sequences in order to assess degrees of identity and/or similarity. These strategies include, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW, etc. When utilizing BLAST and Gapped BLAST programs, default parameters of the respective programs may be used. Alternatively, the practitioner may use non-default parameters depending on his or her experimental and/or other requirements (see for example, the Web site having URL www.ncbi.nlm.nih.gov).

An AMPA receptor or AMPAR is one of a family of ligand-gated ion channels that bind preferentially to α-amino-3-hydroxy-5-methyl-4-isoxazolepropionic acid and that mediate the vast majority of excitatory neurotransmission in the brain (Kim K. S. et al., J. Neurosci., 2010 January; 30(3):1064-72.). The receptors include subunits reported in the literature as GluR1 (GRIA1), GluR2 (GRIA2), GluR3 (GRIA3), and GluR4, alternatively called GluRA-D2 (GRIA4), which combine to form tetramers and perform distinct pharmacological functions. GenEMBL Accession Nos. have been reported for GluR1 (157354), GluR2A (A46056), GluR3 (X82068), and GluR4 (U16129) and are described in WO 94/06345 to Dambinova. An AMPAR receptor peptide refers to a full length AMPA receptor protein, a peptide fragment of the naturally or synthetically occurring full length AMPA receptor, or an analogue or isoform thereof. An GluR peptide thus includes the full length GluR1-3 subunits, in addition to fragments, analogs and derivatives thereof. Similarly, an GluR1-3 peptide means the full length naturally occurring GluR1-3 peptide subunits, or a fragment, analog or derivative thereof. The N-terminal domain of AMPA peptides refers to the amino acid N-terminal domain fragment of the full length peptide, or a fragment, analog or derivative thereof, typically about 40 or 50 amino acids long, but as much as 150, 200 or 300 amino acids long, as described in WO 94/06345 to Dambinova. The GluR1 receptor subunit amino acid sequence is provided in SEQ ID NO:1. The GluR2 amino acid sequence is provided in SEQ ID NO:2. The GluR3 amino acid sequence is provided in SEQ ID NO:3.

A kainate receptor or KAR is one of a family of non-NMDA ionotropic receptors that bind preferentially to kainate and postsynaptic KARs mediate the excitatory neurotransmission in CNS (Dingledine R, et al., Pharmacol. Rev., 1999, 51 (1): 7-61). The receptors include subunits reported in the literature as GluR5 (GRIK1), GluR6 (GRIK2), GluR7 (GRIK3), KA1 (GRIK4), and KA2 (GRIK5), which is permeable to sodium and potassium ions. GenEMBL Accession Nos. have been reported for GluR5 (U16125), GluR6 (U16126), GluR7 (U16127), KA1 (S67803), and KA2 (540369). A KAR receptor peptide refers to a full length KAR receptor protein, a peptide fragment of the naturally or synthetically occurring full length KAR receptor, or an analogue or isoform thereof. A KAR peptide thus includes the full length GluR6/7 subunits, in addition to fragments, analogs and derivatives thereof. Similarly, an GluR6/7 peptide means the full length naturally occurring GluR6/7 peptide subunits, or a fragment, analog or derivative thereof. The N-terminal domain of AMPA peptides refers to the amino acid N-terminal domain fragment of the full length peptide, or a fragment, analog or derivative thereof, typically about 40 or 50 amino acids long, but as much as 150, 200 or 300 amino acids long, as described in WO 94/06345 to Dambinova. The amino acid sequence of GluR6 is provided in SEQ ID NO:4. The amino acid sequence of GluR7 is provided in SEQ ID NO:5.

As used herein, the terms inhibitor and peptide inhibitor, when used in the context of modulating a binding interaction (such as the binding of a glutamate, AMPA, kainate and polyamine domain sequences to the N-terminal fragment of natural or synthetic AMPA receptor sequence), are used interchangeably and refer to an agent that reduces the binding of the, e.g., N-terminal fragment of natural or synthetic AMPA receptor sequence and the, e.g., domain peptide.

An analogue of a peptide means a peptide that contains one or more amino acid substitutions, deletions, additions, or rearrangements. For example, it is well known in the art of protein biochemistry that an amino acid belonging to a grouping of amino acids having a particular size or characteristic (such as charge, hydrophobicity, and hydrophilicity) can often be substituted for another amino acid without altering the activity of the protein, particularly in regions of the protein that are not directly associated with biological activity. Thus, an analogue of an AMPAR/KAR peptide is useful in the present invention if it includes amino acid substitutions, deletions, additions or rearrangements at sites such that antibodies raised against the analogue are still specific against the AMPAR/KAR peptide.

An AMPAR/KAR analogue or mutant as used in this document refers to a sequence that has at least 80% amino acid identity with a AMPAR/KAR peptide, although it could also contain at least 85%, 90%, or 95% identity. Amino acid identity is defined by an analogue comparison between the analogue or mutant and naturally occurring AMPAR/KAR peptide. The two amino acid sequences are aligned in such a way that maximizes the number of amino acids in common along the length of their sequences; gaps in either or both sequences are permitted in making the alignment in order to maximize the number of common amino acids. The percentage amino acid identity is the higher of the following two numbers: (1) the number of amino acids that the two peptides have in common with the alignment, divided by the number of amino acids in the AMPAR/KAR analogue, multiplied by 100, or (2) the number of amino acids that the two peptides have in common with the alignment, divided by the number of amino acids in naturally occurring AMPAR/KAR peptide, multiplied by 100.

AMPAR/KAR derivatives include naturally occurring AMPAR/KAR and AMPAR/KAR analogues and fragments thereof that are chemically or enzymatically derivatized at one or more constituent amino acids, including side chain modifications, backbone modifications, and N- and C-terminal modifications, by for example acetylation, hydroxylation, methylation, amidation, phosphorylation or glycosylation. The term also includes AMPAR/KAR salts such as zinc AMPAR/KAR and ammonium AMPAR/KAR.

A protein or peptide is optionally measured directly in the sense that the protein or peptide is itself measured in the biological sample, as opposed to some other indirect measure of the protein or peptide such as antibodies to the protein or peptide, or cDNA or mRNA associated with the expression of the protein or peptide.

The term antibody is used synonymously herein with immunoglobulin. As used herein, the term antibody includes the native antibody, monoclonally generated antibodies, polyclonally generated antibodies, recombinant DNA antibodies, and biologically active analogues, derivatives or fragments of antibodies, such as, for example, Fab′, F(ab′)2 or Fv as well as single-domains and single-chain antibodies. A biologically active derivative of an antibody is included within this definition as long as it retains the ability to bind the specified antigen. Thus, an AMPAR/KAR antibody that binds specifically to an AMPAR/KAR peptide has the ability to bind at least one AMPAR/KAR peptide. Optionally, the immunoglobulins of the current invention are deglycosylated or dephosphorylated or are prepared synthetically without glycosylation or phosphorylation. When ranges are given by specifying the lower end of a range separately from the upper end of the range, it will be understood that the range can be defined by selectively combining any one of the lower end variables with any one of the upper end variables that is mathematically possible.

Provided herein are AMPAR/KAR peptide isoforms released to the bloodstream, and methods for identifying the location in the human body where particular AMPAR/KAR isoforms are expressed. The AMPAR/KAR isoforms have been revealed by HPLC (on the basis of different retention time), electrofocusing (various protein pl) and immunoblot of peptides using monoclonal antibodies to phosphorylated or acetylated isoform peptide fragments. Glutamate receptor isoforms targeted by this invention include recombinant and/or mutant subtypes that are optionally modified through acetylation or phosphorylation.

Compositions, methods and kits are provided for determining the anatomical source of AMPAR/KAR isoform expression in the human body, and the pathological process leading to such over-expression. The location of such over-expression, the pathological process leading to such over-expression, the diseased state associated with such over-expression, are determined by the particular AMPAR/KAR isoform that is over-expressed. The AMPAR/KAR peptides can be used to distinguish between of the tissues, processes, or CNS injury. An AMPAR/KAR peptide refers to a peptide that combines sequences from the GLuR1-3/GluR6-7 receptor subtype. The sequences are preferably antigenic, and preferably derive from the N-terminal domain of the recited non-NMDA receptor subtype. The peptides are preferably less than about 100, 60 or 40 amino acids in length, and greater than about 10, 15 or 20 amino acids. It will of course be understood that analogs of such sequences may also be present in the recombinant peptide. A particularly useful embodiment involves the detection or measurement of GLuR1-3/GluR6-7, insofar as the results can be used to distinguish between CNS injury (traumatic brain or spinal cord injury), when GLuR1-3/GluR6-7 is measured above a designated standard or otherwise detected, and polytrauma and peripheral nervous system injury. Therefore, provided is a method of diagnosing AMPAR/KAR over-expression in a human subject comprising: a) testing in a bodily fluid, directly or indirectly, the amount of one or more recombinant AMPAR/KAR peptide fragments selected from: i) an GLuR1-3/GluR6-7 recombinant peptide or analog thereof; b) optionally comparing said amounts of GLuR1-3/GluR6-7 peptide fragments with designated standards for said recombinant GLuR1-3/GluR6-7 peptide fragments; and c) optionally correlating an excess amount of one or more recombinant GLuR1-3/GluR6-7 peptide fragments with an anatomical location of AMPAR/KAR over-expression in the patient. Said designated standard for GLuR1-3/GluR6-7 preferably refers to a population norm in apparently healthy human subjects, polytrauma and peripheral nervous system injury or a previously recorded value of GLuR1-3/GluR6-7 for said patient.

A control or standard control refers to a sample, measurement, or value that serves as a reference, usually a known reference, for comparison to a test sample, measurement, or value. For example, a test sample can be taken from a subject suspected of having a given disease (e.g., traumatic brain injury or spinal cord injury) and compared to a known normal (non-diseased or non-injured) individual (i.e., a standard control subject). A standard control can also represent an average measurement or value gathered from a population of similar individuals (i.e., standard control subjects) that do not have a given disease (i.e., standard control population), e.g., healthy individuals with a similar medical background, same age, weight, etc. A standard control value can also be obtained from the same individual, e.g., from an earlier-obtained sample from the subject prior to the onset of disease or injury. One of skill will recognize that standard controls can be designed for assessment of any number of parameters (e.g., RNA levels, protein levels, specific cell types, specific bodily fluids, specific tissues, and the like). The designated standard or control may refer to a non-detectable quantity of peptide or analog thereof. Normal population levels for GLuR1-3/GluR6-7 peptides range generally from 0.01 to 1.0 ng/ml of plasma and an upper of lower cutoff may be selected from any figure between these two endpoints including 0.01, 0.025, 0.05, 0.075, 0.1, 0.25, 0.5, 0.75 or 1.0 ng/ml of plasma. Said designated standard or control for GLuR1-3/GluR6-7 preferably refers to a normal population level in apparently healthy human subjects, or a previous recorded value of GLuR1-3/GluR6-7 for said patient or subject. Alternatively, said designated standard may simply refer to a non-detectable quantity of peptide or analog thereof. Normal population levels for GLuR1-3/GluR6-7 peptides range generally from 0.01 to 1.0 ng/ml of plasma and an upper or lower cutoff may be selected from any figure between these two endpoints including 0.01, 0.025, 0.05, 0.075, 0.1, 0.25, 0.5, 0.75 or 1.0 ng/ml of plasma.

Provided herein are isolated recombinant or mutant GLuR1-3/GluR6-7 peptides, and antibodies specific for said recombinant or mutant GLuR1-3/GluR6-7 peptides for detection of said peptides. Optionally, provided are antibodies that are specific for the recombinant or mutant GLuR1-3/GluR6-7 peptides or nucleic acids that encodes the recombinant peptides. Optionally, the antibodies against the peptides or nucleic acids are bound to a diagnostic substrate or an indicator reagent.

The term isolated excludes instances wherein the peptide or antibody may have been separated from other peptide bands, as in gel electrophoresis, but the peptide or antibody has not been physically isolated from the gel or the other peptide bands. Optionally, the peptide is exactly as represented, or an analog thereof, optionally bound through an appropriate linker to a diagnostic substrate (such as a plate, a particle or a bead) or to an indicator reagent. Similarly, the antibodies and peptides are preferably specific for the exact sequences represented (or analogs thereof), and are optionally bound through an appropriate linker to a diagnostic substrate or an indicator reagent. For example, exemplary amino acid sequences for the recombinant GLuR1-3/GluR6-7 peptides discussed herein are set forth below in Table 1.

Provided herein are AMPAR/KAR peptides selected from the group consisting of SEQ ID NO:6, SEQ ID NO:7, SEQ ID NO:8, SEQ ID NO:9 and SEQ ID NO:10. Also provided are peptides having 80, 81, 82, 83, 84, 85, 86, 87, 88, 89, 90, 91, 92, 93, 94, 95, 96, 97, 98, or 99% identity to SEQ ID NO:6, SEQ ID NO:7, SEQ ID NO:8, SEQ ID NO:9 and SEQ ID NO:10.

TABLE 1
AMPA/KAR Peptides.

| Peptide/ | | |
|---|---|---|
| Isoform | Amino Acid Sequence | Structure |
| GluR1/GluR6_7 | LANKYKELPADTKDA 15 | Hippocampus, |
| | (SEQ ID 6) | amygdala |
| GluR2/GluR6_7 | AAEKKQVLPADTKDA 15 | Hypothalamus, |
| | (SEQ ID 7) | thalamus, basal |
| | | nucleus |
| GluR3/GluR6_7 | KHHPLLPADTKDAPLL 16 | Brainstem: |
| | (SEQ ID 8) | midbrain, pons |
| GluR1/GluR6 | YKESGALPADTKDA 14 | Cervical: |
| | (SEQ ID 9) | medulla |
| GluR2/GluR7 | AAEKKQVLIDSDDPLL 16 | Spinal cord: |
| | (SEQ ID 10) | thotacic, |
| | | lumbar |

Provided herein are methods for determining the level of an AMPAR/KAR peptide in a subject. The methods include obtaining a biological sample from the subject and determining a level of one or more AMPAR/KAR peptides selected from the group consisting of GluR1/GluR6_7, GluR2/GluR6_7, GluR3/GluR6_7, GluR1/GluR6, GluR2/GluR7, and any combination thereof in the biological sample. Optionally, the method further includes comparing the level of the one or more AMPAR/KAR peptides to a control. Optionally, the control level of the AMPAR/KAR peptides is between 0.1 ng/ml and 1.0 ng/ml. Optionally, the biological sample is a blood-derived biological sample. Optionally, the step of determining the level of the one or more AMPAR/KAR peptides comprises performing a Northern Blot, RT-PCR, microarray analysis, sequencing, an immunoassay, a Western Blot, an agglutination assay, fluroescence assay or any combination thereof. Optionally, the subject has a mild traumatic brain injury or an incomplete spinal cord injury. Optionally, the subject has a concussion. Optionally, the AMPAR/KAR peptides are selected from the group consisting of SEQ ID NO:6, SEQ ID NO:7, SEQ ID NO:8, SEQ ID NO:9, SEQ ID NO:10 and any combination thereof. Optionally, the level of SEQ ID NO:6, SEQ ID NO:7, SEQ ID NO:8, SEQ ID NO:9, and SEQ ID NO:10 are determined. Optionally, the AMPAR/KAR peptides are peptides having 80, 81, 82, 83, 84, 85, 86, 87, 88, 89, 90, 91, 92, 93, 94, 95, 96, 97, 98, or 99% identity to SEQ ID NO:6, SEQ ID NO:7, SEQ ID NO:8, SEQ ID NO:9, or SEQ ID NO:10.

Biological sample or sample refers to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), perspiration, saliva, cerebrospinal fluid, sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

Thus, the methods can be performed using practically any biological fluid where circulating cerebral AMPA/KAR receptors, or markers of such receptors, are expressed or found, including blood, urine, blood plasma, blood serum, cerebrospinal fluid, saliva, perspiration or brain tissue. Optionally, the biological fluid is plasma or serum. Optionally, the plasma or serum is undiluted or diluted to a ratio of about 1:1 to 1:50.

The terms subject, patient, individual, etc. are not intended to be limiting and can be generally interchanged. That is, an individual described as a patient does not necessarily have a given disease, but may be merely seeking medical advice. As used throughout, a subject can be a vertebrate, more specifically a mammal (e.g., a human, horse, cat, dog, cow, pig, sheep, goat, mouse, rabbit, rat, and guinea pig), birds, reptiles, amphibians, fish, and any other animal. The term does not denote a particular age or sex. Thus, adult and newborn subjects, whether male or female, are intended to be covered. As used herein, patient, individual and subject may be used interchangeably and these terms are not intended to be limiting. That is, an individual described as a patient does not necessarily have a given disease, but may be merely seeking medical advice. The terms patient or subject include human and veterinary subjects.

The methods can be performed using any number of known diagnostic techniques, including direct or indirect ELISA, RIA, immunodot, immunoblot, latex agglutination, lateral flow, fluorescence polarization, and microarray. Optionally, the method is performed using an immobilized solid phase for capturing specific antibodies and measuring the AMPAR/KAR peptide marker. Therefore, the methods can include (a) contacting a biological sample from the patient with an immobilized solid phase comprising an antibody against AMPAR/KAR peptide, for a time sufficient to form a complex between said AMPAR/KAR peptide and AMPAR/KAR antibody in said biological sample; (b) contacting said complex with an indicator reagent attached to a signal-generating compound to generate a signal; and (c) measuring the signal generated. In a preferred embodiment, the indicator reagent comprises chicken anti-human or anti-human IgG attached to horseradish peroxidase.

Optionally, the solid phase is a polymer matrix. Optionally, the polymer matrix is polyacrylate, polystyrene, or polypropylene. In one preferred embodiment the solid phase is a microplate. Optionally, the solid phase is a nitrocellulose membrane or a charged nylon membrane.

Optionally, the method is performed using agglutination. Therefore, the method can include (a) contacting a biological sample from the patient with an agglutinating carrier comprising an antibody against AMPAR/KAR peptide, for a time sufficient to form an agglutination complex between said peptide and AMPAR/KAR antibody in said biological sample; (c) generating a signal from the agglutination; (d) correlating said signal to said levels of one or more markers of peptide. Optionally, the sufficient time is less than 30, 20, 15, 10 or even 5 minutes.

Latex agglutination assays have been described in Beltz, G. A. et al., in Molecular Probes: Techniques and Medical Applications, A. Albertini et al., eds., Raven Press, New York, 1989, incorporated herein by reference in its entirety. In the latex agglutination assay, antibody raised against a particular biomarker is immobilized on latex particles. A drop of the latex particles is added to an appropriate dilution of the serum to be tested and mixed by gentle rocking of the card. With samples lacking sufficient levels of the biomarkers, the latex particles remain in suspension and retain a smooth, milky appearance. However, if biomarkers reactive with the antibody are present, the latex particles clump into visibly detectable aggregates.

An agglutination assay can also be used to detect biomarkers wherein the corresponding antibody is immobilized on a suitable particle other than latex beads, for example, on gelatin, red blood cells, nylon, liposomes, gold (platinum, silver, other metals), magnetic particles, and the like. The presence of antibodies in the assay causes agglutination, similar to that of a precipitation reaction, which can then be detected by such techniques as nephelometry, turbidity, infrared spectrometry, visual inspection, colorimetry, and the like.

The term latex agglutination is employed generically herein to refer to any method based upon the formation of detectable agglutination, and is not limited to the use of latex as the immunosorbent substrate. While preferred substrates for the agglutination are latex based, such as polystyrene and polypropylene, particularly polystyrene, other well-known substrates include beads formed from glass, paper, dextran, and nylon. The immobilized antibodies may be covalently; ionically, or physically bound to the solid-phase immunoadsorbent, by techniques such as covalent bonding via an amide or ester linkage, ionic attraction, or by adsorption. Those skilled in the art will know many other suitable carriers for binding antibodies, or will be able to ascertain such, using routine experimentation.

Conventional methods can be used to prepare antibodies for use in the present invention. For example, by using a peptide of a AMPAR/KAR protein, polyclonal antisera or monoclonal antibodies can be made using standard methods. A mammal, (e.g., a mouse, hamster, or rabbit) can be immunized with an immunogenic form of the peptide which elicits an antibody response in the mammal. Techniques for conferring immunogenicity on a peptide include conjugation to carriers or other techniques well known in the art. For example, the peptide can be administered in the presence of adjuvant. The progress of immunization can be monitored by detection of antibody titers in plasma or serum. Standard ELISA or other immunoassay procedures can be used with the immunogen as antigen to assess the levels of antibodies. Following immunization, antisera can be administered and, if desired, polyclonal antibodies isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art, (e.g., the hybridoma technique originally developed by Kohler and Milstein (Nature 256, 495-497 (1975)) as well as other techniques such as the human B-cell hybridoma technique (Kozbor et al., Immunol. Today 4, 72 (1983)), the EBV-hybridoma technique to produce human monoclonal antibodies (Cole et al. Monoclonal Antibodies in Cancer Therapy (1985) Allen R. Bliss, Inc., pages 77-96), and screening of combinatorial antibody libraries (Huse et al., Science 246, 1275 (1989)). Hybridoma cells can be screened immunochemically for production of antibodies specifically reactive with the peptide and the monoclonal antibodies can be isolated. Therefore, the invention also contemplates hybridoma cells secreting monoclonal antibodies with specificity for NMDAR proteins or fragments as described herein.

In addition, the method can be practiced using commercially available chemiluminescence techniques. For example, the method and kit can employ a two-site sandwich immunoassay using direct chemiluminescent technology, using constant amounts of two monoclonal antibodies. The first antibody, in a fluid reagent, could be an acridinium ester labeled monoclonal mouse anti-human AMPA/KAR receptor peptide BNP (F(ab′)2 fragment specific to a first portion of the peptide. The second antibody, in the solid phase, can be a biotinylated monoclonal mouse anti-human antibody specific to another portion of the peptide, which can be coupled to streptavidin magnetic particles. An immuno-complex is formed by mixing a patient sample and the two antibodies. After any unbound antibody conjugates are washed away, the chemiluminescence of the immuno-complex signal is then be measured using a luminometer.

When the AMPA/KAR receptors are detected indirectly, by measuring the cDNA expression of the AMPA/KAR receptors, the measuring step in the present invention may be carried out by traditional PCR assays such as cDNA hybridization, Northern blots, or Southern blots. These methods can be carried out using oligonucleotides encoding the polypeptide antigens. Thus, the methods of this invention optionally include measuring an increase of AMPAR/KAR cDNA expression by contacting the total DNA isolated from a biological sample with oligonucleotide primers attached to a solid phase, for a sufficient time period. Optionally, AMPAR/KAR cDNA expression is measured by contacting an array of total DNA bound to a solid matrix with a ready-to-use reagent mixture containing oligonucleotide primers for a sufficient time period. Expressed AMPAR/KAR cDNA is revealed by the complexation of the cDNA with an indicator reagent that comprises a counterpart oligonucleotide to the cDNA attached to a signal-generating compound. The signal-generating compound is optionally selected from the group consisting of horseradish peroxidase, alkaline phosphatase, urinase and non-enzyme reagents. The signal-generating compound is most preferably a non-enzyme reagent.

The immunosorbent for measuring levels of peptides can be produced as follows. A fragment of the antibody to respective receptor protein is fixed, preferably by covalent bond or an ionic bond, on a suitable carrier such as polystyrene or nitrocellulose. If the standard polystyrene plate for immunological examinations is employed, it is first subjected to the nitration procedure, whereby free nitrogroups are formed on the plate surface, which are reduced to amino groups and activated with glutaric dialdehyde serving as a linker. Next the thus-activated plate is incubated with about 2 to 50 nM of the target antibody against respective peptide for the purpose of chemically fixing the respective immunogenic fragment of the receptor protein for a time and at a temperature sufficient to assure fixation (i.e., for about 16 hours at 4° C.).

It is also possible to produce the immunosorbent by fixing the respective antibody against fragment of the receptor protein on nitrocellulose strips by virtue of ionic interaction to produce multiple panel of biomarkers. The respective antibody raised against fragment of the receptor protein is applied to nitrocellulose and incubated for 15 min at 37° C. Then nitrocellulose is washed with a 0.5% solution of Tween-20, and the resultant immunosobent is dried at room temperature and stored in dry place for one year period.

The provided methods can include treating the mild traumatic brain injury or incomplete spinal cord injury. Thus, the provided methods can include administering to the subject a pharmaceutical composition comprising a compound having the chemical structure Gly-Xn-Glu, wherein X is an amino acid and n is 2, 3, or 4. Optionally, the compound comprising a chelating agent selected from the group consisting of magnesium, iron and zinc. Thus, provided are compounds that are useful for antagonizing AMPAR/KAR over-expression in the brain and spinal cord induced by mild or incomplete injury. These novel compounds can be defined by the chemical structure (I): Gly-X-Glu (I) wherein: Gly is the residue of glycine and Glu is the residue of glutamic acid; and X is a chain comprising from two to four amino acid residues and an optional chelating agent selected from magnesium, iron and zinc. Optionally, —X— is represented by chemical structure (II): -A-B- (II), wherein A is independently one or more residues of arginine, or lysine or glutamine or glutamic acid; and B is a chain comprising a chelating agent selected from magnesium, iron and zinc. Optionally, A comprises independently residues of lysine or glutamic acid or arginine; and B is a metal chelating agent

Provided are pharmaceutical compositions comprising the compounds and one or more pharmaceutically acceptable excipients. The compositions comprise an amount sufficient to reversible inhibit AMPA/KAR activity in combination with a pharmaceutically acceptable excipient. Provided is a method of inhibiting AMPA/KAR activity comprising administering to a subject a pharmaceutical composition comprising the provided compounds, in an amount sufficient to antagonize AMPA/KAR activity. Optionally, the patient is diagnosed as having mild TBI or incomplete SCI. Suitable dose ranges include, for example, from about 0.01 to about 10.0 mg/day, about 0.05 to about 1.0 mg/day, and from about 0.1 to about 0.5 mg/day. The dose may be administered once, twice or even three times per day, and is preferably administered until a desired level of clinical improvement is observed in the subject to whom the drug is administered.

The compound may be prepared by numerous methods known in the art to synthetic chemists for making and purifying amino acid chelates. Methods are taught, for example, in U.S. Pat. Nos. 4,830,716 and 4,599,152. For example, pharmaceutical grade amino acid and peptide chelates, free of interfering anions, can be made by reacting one or more amino acid or peptides with a metal member selected from the group consisting of elemental metals, metal oxides, metal hydroxides and metal carbonates in an aqueous environment employing at least a two-fold molar excess of amino acid or peptide relative to the metal. The reaction may be carried out in the presence of non-interfering anions such as anions from citric acid, ascorbic acid, acetic acid, carbonic acid and ammonium and alkali metal salts thereof. Where the molecule desired is not perfectly symmetrical (i.e., when there are two different peptides or amino acids chelated to the metal), it is necessary to separate the desired product from the reaction mixture, and such separation is optionally performed using conventional techniques such as high performance liquid chromatography. Separation methods are taught, for example, in Tommel D K L et al., A method for the separation of peptides and α-amino acids; Biochem J. 1968 April; 107(3): 335-340, and in Hill-Cottingham D G et al., Analysis of iron chelates in plant extracts; J Sci of Food and Agr. 2006; 12(1): 69-74.

The compounds may be administered to a subject per se or in the form of a sterile composition or a pharmaceutical composition. Pharmaceutical compositions comprising the compounds of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries that facilitate processing of the active peptides or peptide analogues into preparations that can be used pharmaceutically. Proper formulation is dependent upon the route of administration chosen.

For topical administration, the compounds can be formulated as solutions, gels, ointments, creams, suspensions, etc. as are well-known in the art. Systemic formulations include those designed for administration by injection, e.g., subcutaneous, intravenous, intramuscular, intranasal, intrathecal or intraperitoneal injection, as well as those designed for transdermal, transmucosal, oral or pulmonary administration.

For injection, the compounds can be formulated in aqueous solutions, preferably in physiologically compatible buffers such as Hanks's solution, Ringer's solution, or physiological saline buffer. The solution can contain formulatory agents such as suspending, stabilizing and/or dispersing agents. Alternatively, the compounds can be in powder form for constitution with a suitable vehicle, e.g., sterile pyrogen-free water, before use.

For oral administration or vaccination, the compounds can be readily formulated by combining the active peptides (antibodies) or peptide analogues with pharmaceutically acceptable carriers well known in the art. Such carriers enable the compounds of the invention to be formulated as tablets, pills, draggers, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, and the like. Additionally, flavoring agents, preservatives, coloring agents and the like may be added. For buccal administration, the compounds may take the form of tablets, lozenges, and the like, formulated in conventional manner.

For administration by inhalation, the compounds are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also maybe employed, although usually at the cost of greater toxicity. Additionally, the compounds may be delivered using a sustained-release system, such as semi-permeable matrices of solid polymers containing the therapeutic agent. Various of sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

As the compounds may contain charged side chains or termini, they may be included in any of the above-described formulations as the free bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

The compounds are used in an amount effective to achieve the intended purpose (e.g., treatment of neural injury such as concussion or mild TBI). The therapies are performed by administering the subject drug in a therapeutically effective amount. By therapeutically effective amount is meant an amount effective to ameliorate or prevent the symptoms or to prolong the survival of the subject being treated. Determination of a therapeutically effective amount is well within the capabilities of those skilled in the art, especially in light of the detailed disclosure provided herein. A therapeutic amount or therapeutic concentration of AMPAR/KAR isoforms or antibodies is an amount that reduces binding activity to receptor by at least about 40%, preferably at least about 50%, often at least about 70%, and even as much as at least about 90%. Binding can be measured in vitro (e.g., in an assay) or in situ.

For systemic administration, a therapeutically effective dose can be estimated initially from in vitro assays. For example, a dose can be determined in culture and further assessed in animal models to achieve a circulating concentration range that includes the IC50. Initial dosages can also be estimated from in vivo data, e.g., animal models, using techniques that are well known in the art. One having ordinary skill in the art could readily optimize administration to humans based on animal data. Such information can be used to more accurately determine useful doses in humans.

Dosage amount and interval may be adjusted individually to provide plasma levels of the compounds that are sufficient to maintain therapeutic effect. Usual patient dosages for administration by injection range from about 0.1 to 10 mg/day, preferably from about 0.5 to 1 mg/day. Therapeutically effective serum levels may be achieved by administering multiple doses each day. For usual peptide/antibodies therapeutic treatment of neural injury within 6 hours of event is typical.

In cases of local administration or selective uptake, the effective local concentration of the compounds may not be related to plasma concentration and should be optimized therapeutically effective local dosages without undue experimentation. The amount of compound administered will, of course, be dependent on the subject being treated, on the subject's weight, the severity of the affliction or injury, the manner of administration and the judgment of the prescribing physician.

The therapy may be repeated intermittently while symptoms are detectable or even when they are not detectable. The therapy may be provided alone or in combination with other drugs. Preferably, a therapeutically effective dose of the compounds described herein will provide therapeutic benefit without causing substantial toxicity.

Toxicity of the compounds described herein can be determined by standard pharmaceutical procedures in experimental animals, e.g., by determining the LD50 (the dose lethal to 50% of the population) or the LD100 (the dose lethal to 100% of the population). The dose ratio between toxic and therapeutic effect is the therapeutic index. Compounds which exhibit high therapeutic indices are preferred. The data obtained from these cell culture assays and animal studies can be used in formulating a dosage range that is not toxic for use in human. The dosage of the compounds described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the subject's condition. See, e.g., Fingl et al., 1975, In: The Pharmacological Basis of Therapeutics, Ch. 1, p. 1.

Provided herein are diagnostic kits or panel of biomarkers. Also provided are the use of the multiple markers in diagnosing various pathological conditions and anatomical locations associated with over-expression of AMPA/KAR receptors. Such kits, biomarkers and methods are optionally used or carried out by measuring recombinant or mutant peptides. The kit optionally allows one to test for the recombinant or mutant peptides. Optionally, provided is a kit comprising recombinant AMPAR/KAR peptide fragments, or antibodies that specifically bind to recombinant AMPAR/KAR peptide fragments, or nucleic acids that encode recombinant AMPAR/KAR peptide fragments, each bound to a diagnostic substrate or indicator reagent, wherein said recombinant AMPAR/KAR peptide fragments are a GLuR1-3/GluR6-7 recombinant peptide or analog thereof.

Provided are kits including a binding agent capable of binding to a substance within a biological sample from a subject that has a mild traumatic brain injury or an incomplete spinal cord injury, wherein said substance is an AMPAR/KAR peptide selected from the group consisting of SEQ ID NO:6, SEQ ID NO:7, SEQ ID NO:8, SEQ ID NO:9, SEQ ID NO:10, and any combination thereof, and a detecting reagent or a detecting apparatus capable of detecting binding of said binding agent to said substance. Optionally, the binding agents are antibodies or primers. Optionally, the kit comprises binding agents binding SEQ ID NO:6, SEQ ID NO:7, SEQ ID NO:8, SEQ ID NO:9, and SEQ ID NO:10. Optionally, the kit includes AMPAR/KAR peptides selected from the group consisting of SEQ ID NO:6, SEQ ID NO:7, SEQ ID NO:8, SEQ ID NO:9, SEQ ID NO:10, and any combination thereof, and a detecting reagent or a detecting apparatus capable of detecting antibodies to SEQ ID NO:6, SEQ ID NO:7, SEQ ID NO:8, SEQ ID NO:9, SEQ ID NO:10, or any combination thereof. Optionally, the antibodies are detected in a blood-derived biological sample from a subject. Optionally, the kit comprises SEQ ID NO:6, SEQ ID NO:7, SEQ ID NO:8, SEQ ID NO:9 and SEQ ID NO:10.

Kits can also include components for comparing results such as a suitable control sample, for example, a positive and/or negative control. The kit can also include a collection device for collecting and/or holding the sample from the subject. The collection device can include a sterile swab or needle (for collecting blood), and/or a sterile tube (e.g., for holding the swab or a bodily fluid sample). Optionally, the provided kits include instructions for use.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the claims below.

### Examples

### Example 1. Detection of AMPAR/KAR Peptides in Athletes

Athletes (n=33) with clinically defined single or multiple concussions were compared to 91 age and gender matched controls without a history of concussion. AMPAR/KAR peptide values of 0.05-0.90 ng/mL for controls and 1.1-7.4 ng/mL for concussions were found. The biomarkers sensitivity of 89-91% and a specificity of 90-93% (1.0 ng/mL cut off) to assess concussions were calculated. Poorer ImPACT scores correlated with abnormal levels of the biomarkers. In athletes with multiple concussions, increased AMPAR/KAR peptides values (4.0-9.0 ng/mL) were associated with minor findings on 3T magnetic resonance imaging.

### Example 2. Detection of AMPAR/KAR Peptides in Male Student

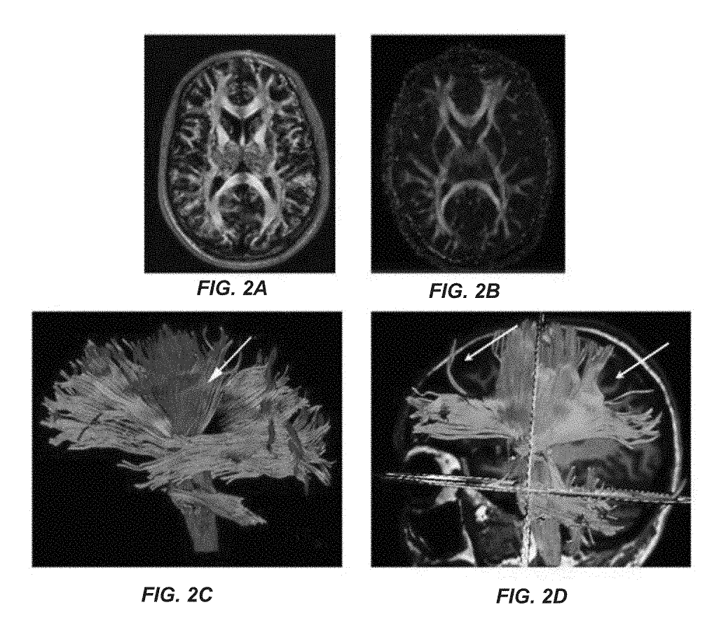

A 22-year-old male student presented with a history of semi-acute concussion due to impact during ice hockey game. After temporary removal from the game, he passed a sideline evaluation and returned to the game. The next day neurological symptoms manifested including headaches, impaired balance, nausea, and sensitivity to light and noise, that persisted for 4 weeks with added sleeplessness. 3T DTI scans for this subject showed significant white matter changes with decreased fractional anisotropy (FA) compared to non-injured subject scan (FIG. 2). There were a number of subcortical white matter fiber tracts absent in frontal and parietal areas of brain indicating the severity of concussion that supported by abnormal values of AMPAR/KA peptides (4.8-5.5 ng/ml).

### Example 3. Detection of AMPAR/KAR Peptides in Military Personnel

AMPA/KAR peptides were detected in active duty military personnel (37M/16F, 23.0±1.2 y.o., 1 week after blast injury, GCS=13-15) as a component of post-military deployment mild TBI screening. There were impaired venous circulation in cervical areas defined by dopplerography yielding an optimal cut off value of 1.0 ng/mL (85% sensitivity, 86% specificity), at which a positive predictive value of 90% was achieved. A clinical study conducted with civilians who sustained mild TBI (25M/20F, 30.1±3.0 y.o., GCS=13-15) and admitted to the emergency department within 24 hours after the impact due to violence-related events, motor vehicle crashes, and incidental falls showed AMPA/KAR peptide sensitivity of 83% and specificity of 85% (cut-off of 1.0 ng/mL), with a significant positive likelihood ratio of 9.5 to assess severe concussions.

### Example 4. Neuroprotective Effects of Gafargin

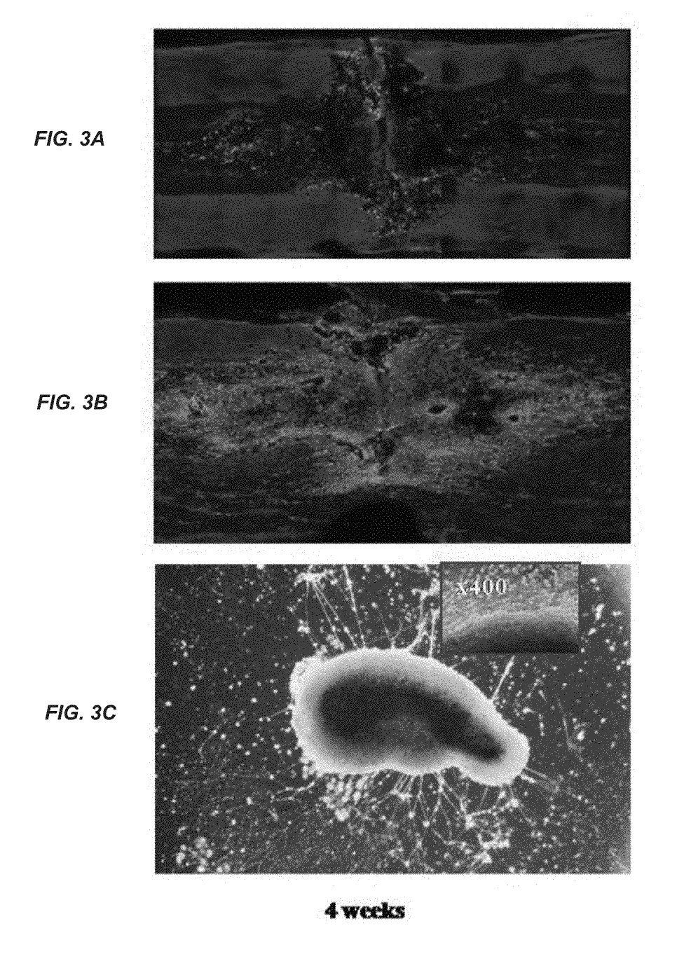

The neuroprotective effects of Gafargin (the commercial name for the composition comprising the peptide Gly-Arg-Glu plus the chealating agent iron (Fe) (Grace Laboratories, LLC, Atlanta, Ga.)) were studied in rats. Gafargin or placebo was injected intraventricularly before incomplete spinal cord injury. The pretreatment with Gafargin (1 μg in 5 μl solution (i.e., 0.3 μM)) generated 40-45% less axonal dieback (FIG. 3A, B). Pretreatment with the peptide did not alter cerebral blood flow or physiological parameters, however decreased the level of GluR1-3/GluR6-7 peptide levels close to that for placebo rats with incomplete spinal cord injury. Additionally, Gafargin demonstrated neurotrophic effect in hippocampal slices when added to medium (FIG. 3C).

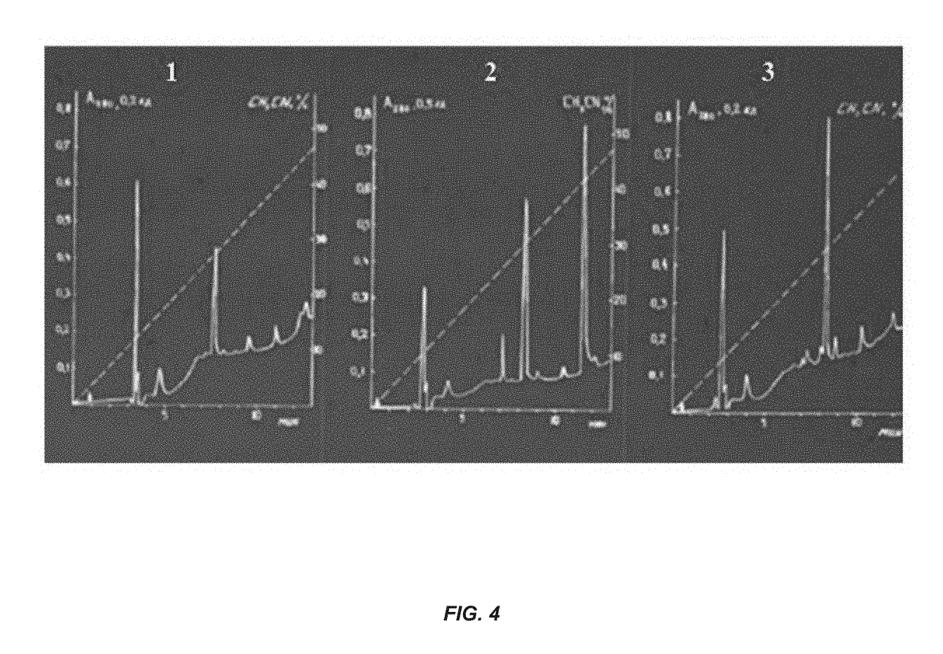

Thirty-three (33) persons (21.0±2.2 y.o.) with symptoms of facial weakness, confusion, and imbalance after mild TBI were enrolled for the study. Ten persons out of 33 received placebo, while remaining 23 subjects had intranasal drops of Gafargin (0.1 mg/day) administered daily for 5 days (Dambinova, Korol'kov in Human Brain and Nerves Therapeutic Electrical Stimulation ed. N. P. Bechtereva, Soya Publishing, Inc., Vladimir, Russia, 2008, p. 346). The efficacy of Gafargin was assessed by mini-mental state examination (MMSE) scores and using GluR1-3/GluR6-7 peptide blood assay. The Gafargin increased total MMSE scores to normal values (from 25 to 30) in the group treated by Gafargin vs. placebo (p<0.05). A normalization was done of GluR1-3/GluR6-7 peptide ratios in cerebrospinal fluid (CSF) (FIG. 4) and in plasma of treated subjects (from 2.5-3.7 ng/ml to 1.1-1.2 ng/ml). Compared to placebo, the third day of gafargin treatment was accompanied with diminishing neurological symptoms.

What is claimed is:

1. A method for determining the level of an AMPAR/KAR peptide in a subject, the method comprising:
(a) obtaining a biological sample from the subject; and
(b) determining a level of one or more AMPAR/KAR peptides selected from the group consisting of GluR1/GluR6_7, GluR2/GluR6_7, GluR3/GluR6_7, GluR1/GluR6, GluR2/GluR7, and any combination thereof in the biological sample.

2. The method of claim 1, further comprising comparing the level of the one or more AMPAR/KAR peptides to a control.

3. The method of claim 2, wherein the control level of the AMPAR/KAR peptides is between 0.1 ng/ml and 1.0 ng/ml.

4. The method of claim 1, wherein the biological sample is a blood-derived biological sample.

5. The method of claim 1, wherein the step of determining the level of the one or more AMPAR/KAR peptides comprises performing a Northern Blot, RT-PCR, microarray analysis, sequencing, an immunoassay, a Western Blot, an agglutination assay, fluorescence assay or any combination thereof.

6. The method of claim 1, wherein the step of determining the level of the one or more AMPAR/KAR peptides comprises detecting, in the biological sample, the level of one or more antibodies to the AMPAR/KAR peptides.

7. The method of claim 6, wherein the control level of the antibodies to the AMPAR/KAR peptides is between 0.1 ng/ml and 10.0 ng/ml.

8. The method of claim 1, wherein the subject has a mild traumatic brain injury or an incomplete spinal cord injury.

9. The method of claim 1, further comprising administering to the subject a pharmaceutical composition comprising a compound having the chemical structure Gly-Xn-Glu, wherein X is an amino acid and n is 2, 3, or 4.

10. The method of claim 9, wherein the compound comprising a chelating agent selected from the group consisting of magnesium, iron and zinc.

11. The method of claim 1, wherein the AMPAR/KAR peptides are selected from the group consisting of SEQ ID NO:6, SEQ ID NO:7, SEQ ID NO:8, SEQ ID NO:9, SEQ ID NO:10 and any combination thereof.

12. The method of claim 1, wherein the level of SEQ ID NO:6, SEQ ID NO:7, SEQ ID NO:8, SEQ ID NO:9, and SEQ ID NO:10 are determined.

13. A kit comprising
(a) a binding agent capable of binding to a substance within a biological sample from a subject that has a mild traumatic brain injury or an incomplete spinal cord injury, wherein said substance is an AMPAR/KAR peptide selected from the group consisting of SEQ ID NO:6, SEQ ID NO:7, SEQ ID NO:8, SEQ ID NO:9, SEQ ID NO:10, and any combination thereof; and
(b) a detecting reagent or a detecting apparatus capable of detecting binding of said binding agent to said substance.

14. The kit of claim 13, wherein the binding agents are antibodies or primers.

15. The kit of claim 13, wherein the kit comprises binding agents binding SEQ ID NO:6, SEQ ID NO:7, SEQ ID NO:8, SEQ ID NO:9, and SEQ ID NO:10.

16. A kit comprising
(a) AMPAR/KAR peptides selected from the group consisting of SEQ ID NO:6, SEQ ID NO:7, SEQ ID NO:8, SEQ ID NO:9, SEQ ID NO:10, and any combination thereof; and
(b) a detecting reagent or a detecting apparatus capable of detecting antibodies to SEQ ID NO:6, SEQ ID NO:7, SEQ ID NO:8, SEQ ID NO:9, SEQ ID NO:10, or any combination thereof.

17. The kit of claim 16, wherein the antibodies are detected in a blood-derived biological sample from a subject.

18. The kit of claim 16, wherein the kit comprises SEQ ID NO:6, SEQ ID NO:7, SEQ ID NO:8, SEQ ID NO:9 and SEQ ID NO:10.


SEQUENCE LISTING (10 sequences)

SEQ ID NO: 1 - protein, 518 aa, homo sapiens
ANFPNNIQIGGLFPNQQSQEHAAFRFALSQLTEPPKLLPQIDIVNISDSFEMTYRFCSQFSKGVYAIFGFYERRTVNMLTSFCGALHVCFITPSFPVDTSNQFVLQLRPELQDALISIIDHYKWQKFVYIYDADRGLSVLQKVLDTAAEKNWQVTAVNILTTTEEGYRMLFQDLEKKKERLVVVDCESERLNAILGQIIKLEKNGIGYHYILANLGFMDIDLNKFKESGANVTGFQLVNYTDTIPAKIMQQWKNSDARDHTRVDWKRPKYTSALTYDGVKVMAEAFQSLRRQRIDISRRGNAGDCLANPAVPWGQGIDIQRALQQVRFEGLTGNVQFNEKGRRTNYTLHVIEMKHDSIRKIGYWNEDDKFVPAATDAQAGGDNSSVQNRTYIVTTILEDPYVMLKKNANQFEGNDRYEGYCVELAAEIAKHVGYSYRLEIVSDGKYGARDPDTKAWNGMVGELVYGRADVAVAPLTITLVREEVIDFSKPFMSLGISIMIKKPQKSKPGVFSFLDPLA

SEQ ID NO: 2 - protein, 463 aa, homo sapiens
VSSNSIQIGGLFPRGADQEYSAFRVGMVQFSTSEFRLTPHIDNLEVANSFAVTNAFCSQFSRGVYAIFGFYDKKSVNTITSFCGTLHVSFITPSFPTDGTHPFVIQMRPDLKGALLSLIEYYQWDKFAYLYDSDRGLSTLQAVLDSAAEKKWQVTAINVGNINNDKKDEMYRSLFQDLELKKERRVILDCERDKVNDIVDQVITIGKHVKGYHYIIANLGFTDGDLLKIQFGGANVSGFQIVDYDDSLVSKFIERWSTLEEKEYPGAHTTTIKYTSALTYDAVQVMTEAFRNLRKQRIEISRRGNAGDCLANPAVPWGQGVEIERALKQVQVEGLSGNIKYDQNGKRINYTINIMELKTNGPRKIGYWSEVDKMVVTLTELPSGNDTSGLENKTVVVTTILESPYVMMKKNHEMLEGNERYEGYCVDLAAEIAKHCGFKYKLTIVGDGKYGARDADTKIWNGM

SEQ ID NO: 3 - protein, 524 aa, homo sapiens
GFPNTISIGGLFMRNTVQEHSAFRFAVQLYNTNQNTTEKPFHLNYHVDHLDSSNSFSVTNAFCSQFSRGVYAIFGFYDQMSMNTLTSFCGALHTSFVTPSFPTDADVQFVIQMRPALKGAILSLLGHYKWEKFVYLYDTERGFSILQAIMEAAVQNNWQVTARSVGNIKDVQEFRRIIEEMDRRQEKRYLIDCEVERINTILEQVVILGKHSRGYHYMLANLGFTDILLERVMHGGANITGFQIVNNENPMVQQFIQRWVRLDEREFPEAKNAPLKYTSALTHDAILVIAEAFRYLRRQRVDVSRRGSAGDCLANPAVPWSQGIDIERALKMVQVQGMTGNIQFDTYGRRTNYTIDVYEMKVSGSRKAGYWNEYERFVPFSDQQISNDSASSENRTIVVTTILESPYVMYKKNHEQLEGNERYEGYCVDLAYEIAKHVRIKYKLSIVGDGKYGARDPETKIWNGMVGELVYGRADIAVAPLTITLVREEVIDFSKPLMSLGISIMIKKPQKSKPGVFSFLDPLA

SEQ ID NO: 4 - protein, 528 aa, homo sapiens
HVLRFGGIFEYVESGPMGAEELAFRFAVNTINRNRTLLPNTTLTYDTQKINLYDSFEASKKACDQLSLGVAAIFGPSHSSSANAVQSICNALGVPHIQTRWKHQVSDNKDSFYVSLYPDFSSLSRAILDLVQFFKWKTVTVVYDDSTGLIRLQELIKAPSRYNLRLKIRQLPADTKDAKPLLKEMKRGKEFHVIFDCSHEMAAGILKQALAMGMMTEYYHYIFTTLDLFALDVEPYRYSGVNMTGFRILNTENTQVSSIIEKWSMERLQAPPKPDSGLLDGFMTTDAALMYDAVHVVSVAVQQFPQMTVSSLQCNRHKPWRFGTRFMSLIKEAHWEGLTGRITFNKTNGLRTDFDLDVISLKEEGLEKIGTWDPASGLNMTESQKGKPANITDSLSNRSLIVTTILEEPYVLFKKSDKPLYGNDRFEGYCIDLLRELSTILGFTYEIRLVEDGKYGAQDDANGQWNGMVRELIDHKADLAVAPLAITYVREKVIDFSKPFMTLGISILYRKPNGTNPGVFSFLNPLSP

SEQ ID NO: 5 - protein, 384 aa, homo sapiens
HVIRIGGIFEYADGPNAQVMNAEEHAFRFSANIINRNRTLLPNTTLTYDIQRIHFHDSFEATKKACDQLALGVVAIFGPSQGSCTNAVQSICNALEVPHIQLRWKHHPLDNKDTFYVNLYPDYASLSHAILDLVQYLKWRSATVVYDDSTGLIRLQELIMAPSRYNIRLKIRQLPIDSDDSRPLLKEMKRGREFRIIFDCSHTMAAQILKQAMAMGMMTEYYHFIFTTLDLYALDLEPYRYSGVNLTGFRILNVDNPHVSAIVEKWSMERLQAAPRSESGLLDGVMMTDAALLYDAVHIVSVCYQRAPQMTVNSLQCHRHKAWRFGGRFMNFIKEAQWEGLTGRIVFNKTSGLRTDFDLDIISLKEDGLEKVGVWSPADGLNIT

SEQ ID NO: 6 - protein, 15 aa, homo sapiens
LANKYKELPADTKDA

SEQ ID NO: 7 - protein, 15 aa, homo sapiens
AAEKKQVLPADTKDA

SEQ ID NO: 8 - protein, 16 aa, homo sapiens
KHHPLLPADTKDAPLL

SEQ ID NO: 9 - protein, 14 aa, homo sapiens
YKESGALPADTKDA

SEQ ID NO: 10 - protein, 16 aa, homo sapiens
AAEKKQVLIDSDDPLL